(19) United States
(12) Patent Application Publication
(10) Pub. No.: US 2005/0064383 A1
(43) Pub. Date: Mar. 24, 2005

(54) Methods and apparatus for producing gender enriched sperm

(76) Inventors: James K. Bashkin, St .Louis, MO (US); Bradley A. Didion, Washington, MO (US); Scott S. Woodard, Manchester, MO (US)

Correspondence Address: Nancy Huelskamp;Monsato Company E2NA, 800 North Lindbergh Boulevard, St Louis, MO, 63167, US

(21) Appl. No.: 10/483,151

(22) PCT Filed: Nov. 21, 2001
(86) PCT No.: PCT/US01/43359

Related U.S. Application Data
(60) Provisional application No. 60/252,796, filed on Nov. 22, 2000.

(51) Int. Cl. A61K 35/52; C12Q 1/00

(52) U.S. Cl. 435/4; 424/561

(57) ABSTRACT

Sperm in semen are sorted by fluorescence-activated cell sorting into gender-enriched populations enriched in X-chromosome or Y-chromosome bearing sperm by use of a fluorescent quantitative DNA-binding vital stain.

### FIELD OF THE INVENTION

The invention relates to methods, compositions of matter, and apparatus for sorting sperm to produce subpopulations enriched in sperm carrying chromosome determinants for male or female offspring, hereinafter referred to as gender-enriched sperm (or semen) or GES.

### BACKGROUND OF THE INVENTION

Artificial insemination is widely used in animal husbandry, for example, with economically important mammals such as cattle, pigs, horses, sheep, goats and other mammals. Likewise, in vitro fertilization and embryo transfer technology also have increasing application in species where the value of individual offspring is sufficiently high. Both of these techniques also have human applicability.

It is frequently desired to produce offspring of a predetermined sex or sex ratio, for example, female bovines for milk production or breeding, male bovines and female porcines for meat production The simplest and most economically feasible way preferentially to produce offspring of a predetermined sex or sex ratio would be a high-throughput system for producing gender enriched sperm or semen (GES) which could then be used for artificial insemination (Al) or in vitro fertilization (IVF).

Although there are reports that sperm may be distinguishable based on sex-specific surface antigens, it is generally considered that sperm nearly completely or perhaps completely lack any phenotypic sex-specific character. As a result, current efforts for producing GES in mammalian species rely on techniques responsive to the quantitatively different levels of DNA in male and female sperm in mammalian species. Since, for example, total DNA in mammalian Y-chromosome bearing sperm typically is 2.5 to 5% total DNA less than total DNA in mammalian X-chromosome bearing sperm, this difference has been used to separate sperm into GES using a DNA vital stain comprising a fluorochrome that readily permeates the cell membranes and relatively nonspecifically and uniformly binds to the DNA without unacceptably damaging the viability of the sperm (quantitative DNA vital binding stain or QDVS). The labeled sperm can then be sorted, for example, using ultraviolet laser based cell cytometry to distinguish the resulting quantitative differences in fluorescence between male and female chromosome bearing sperm and to produce GES. Exemplary of patent literature in this area are; Johnson et al., U.S. Pat. No. 5,135,759, and Rens et al., U.S. Pat. No. 5,985,216, which are hereby incorporated by reference for description of methods, compositions of matter and apparatus for producing GES known in the art. However, the methodology of Johnson et al. requires use of a bisbenzimide stain (Hoechst H 33342 fluorochrome (available from Calbiochem-Behring Co., La Jolla, Calif.), at relatively high temperatures to achieve relatively short staining times. According to Johnson et al., for example, incubation for 1 hr at 35° C. was found to be acceptable, and ranges of 30° C. to 39° C. were also stated to be effective requiring corresponding incubation times from 1.5 to 1 hour (the incubation period being less at higher temperatures). However, the use of temperatures in the range of 30° C. to 39° C. in the presence of a QDVS followed by ultraviolet laser based flow cytometry introduces a number of difficulties and disadvantages into the process which begins at semen collection and ends at fertilization which can reduce sperm viability and the efficiency (purity) of sorting sperm into GES.

Prior to the work represented in the Johnson et al. and Rens et al. patents, other less successful or failed efforts had also been made. Some of these used dyes or stains which are only capable of entering permeabilized or dead cells and which are not effective vital stains for sperm, including acridine orange and derivatives thereof such as ethidium bromide, mithramycin or combinations thereof, and further including DAPI (4,6diamidino-2-phenylindole).

Referring now to GB 2 145 112 A, that document purports to describe a method for staining sperm using Hoechst 33342 dye and then sorting the sperm ultimately into two populations AI and AII of motile sperm with the AD population having a fluorescence about 15% greater than that of the AI population. It is well known that the difference in fluorescence between two populations of sperm fully separated on the basis of sex should be on the order of about 3 to about 5% (3.0% for rabbit, 3.6% for boar, 3.8% for bull, and 4.2% for ram sperm). Perhaps for this reason, GB 2 145 112 A2 is able only to speculate on the significance of the difference in fluorescence between the two subpopulations: “The subpopulations (AI and AII) may reflect spermatozoa at distinct stages of late maturation or the difference between X- and Y-chromosome bearing spermatozoa.” For various reasons, however, it is clear to persons skilled in the art that in any event that GB 2 145 112 A did not accomplish separation into subpopulations of 90% or more X- OR Y-bearing sperm.

In addition to the Johnson et al. and Rens et al. patents cited above, patent literature relevant to GES includes U.S. Pat. No. 6,263,745 B1, WO 01/37655, U.S. Pat. No. 6,149,867, U.S. Pat. No. 6,071,689, U.S. Pat. No. 4,362,246 and WO 99/33956. These patents and patent applications and those of Johnson et al. and Rens et al. are incorporated herein by reference as describing methods, compositions of matter and apparatus for handling and producing GES known to those skilled in the art.

Notwithstanding the above-described systems, there remains an urgent need for new and improved GES production and handling methods and apparatus that results in GES having advantageous viability, motility and integrity.

### SUMMARY OF THE INVENTION

If GES is to become widely used in animal husbandry, methodologies must be developed which take into account the effects on sperm of the entire sequence of collecting sperm and preparing and using GES. For example, sperm might be collected from a donor animal in a breeder herd maintained at a remote location, prepared for transport at the point of collection in a processing facility optionally with QDVS staining, transported under controlled conditions to a sorting facility, optionally with QDVS staining to occur at the sorting facility, sorted into GES, prepared optionally with freezing for shipping, shipped under controlled conditions to a breeding facility, thawed and used. At several or most of these steps, as practiced in the prior art, the sperm will be exposed to changes in temperature and to changes in the fluid environment including pH changes or other environmental conditions which will individually or cumulatively affect staining and separation efficiency and viability (motility) of the sperm.

We have found in staining at a temperature in the range of about 17° C. to less than about 30° C. that the pH of the fluid environment to which the sperm are exposed during staining has a significant influence over the period of time required for uniform staining sufficient for production of GES. Accordingly, we have found that a prolonged period of staining, such as during transit from a collection facility to a sorting facility, can be used, at effective temperatures between about the thermotropic phase transition temperature Tₘ of the membranes of the sperm being sorted up to less than about 30° C. and at an effective pH between about 6.8 and about 7.6, to reduce or eliminate the time required for higher temperature incubation with stain. The lower temperatures (compared to prior art techniques) are also believed to provide advantageous effects on sperm orientation during sorting.

According to the invention herein, there are provided methods which avoid the high temperature QDVS stain incubation step of Johnson et al. and advantageously conduct all processing steps between collection and providing GES to the site of ultimate use within a narrower range of temperatures (from about 17° C., or even lower depending on species, to less than about 30° C.) that are advantageous and beneficial to high levels of viability (motility), and of separation efficiency (purity) of the resulting GES. According to an aspect of the invention, incubation with QDVS occurs at least in part at a pH in the range of about 7.1 to about 7.6, or according to another aspect in the range of about 6.8 to about 7.6. According to a further aspect of the invention, a QDVS is used which permits visible light-based flow cytometry (as compared to an ultraviolet-based flow cytometry system) to be used, further reducing damage to the sperm and reducing the costs of flow cytometry equipment.

According to various other aspects, the invention relates to process and apparatus for producing GES (gender enriched semen) comprising providing a suspension of viable sperm produced from collected semen ejaculate that is extended and transported to a sorting facility, staining the sperm using a QDVS (quantitative DNA vital Stain), producing at least one of X-enriched and Y-enriched GES based on the extent of QDVS staining of DNA, collecting the resulting GES, and apportioning the collected GES into dosage quantities for use or shipment. In one aspect, all of the steps occur at a temperature between a lower temperature at which the sperm remain mostly viable and an upper temperature of less than about 30° C. According to other aspects, the upper temperature may range on upwards to less than about 39° C. and the staining, producing and collecting steps all occur in the presence of media comprising a buffer system and further optionally including other components effective for maintaining viability of at least a portion of the semen, wherein all of the media comprise the same or substantially the same buffer systems. According to a further aspect, even the providing and partitioning steps also occur in the presence of such media.

According to yet further aspects of the invention, the step of producing involves the use of a Fluorescence-Activated Flow Sorter (FACS) to sort the sperm based on extent of DNA staining where the sheath fluid also is such a medium as previously described, or where the QVDS is a visible-light stimulated QVDS, or where the QVDS is a visible light excited QVDS and visible light irradiation is used for the producing step.

The invention will be further described in detail and in terms of certain preferred embodiments; however, other uses, applications and embodiments will be apparent to, or readily developed without undue experimentation by, those skilled in the art from the following detailed description and the examples.

### BRIEF DESCRIPTION OF THE DRAWINGS

Turning now to the Drawings, FIG. 1 is a block diagram illustrating methods according to the invention wherein certain steps are preferably conducted using a sperm maintenance media comprising the same or substantially the same buffer system and wherein all of steps 20-50 can preferably be conducted in a relatively narrow temperature range from about the thermotropic phase transition temperature of the sperm being sorted up to less than about 30° C., and where optionally a FACS step is conducted using visible light laser stimulation of an effective visible-light stimulated QDVS fluorophore.

FIG. 2 schematically illustrates preferred flow cytometric means and methods for separating living cells and cell clusters according to an aspect of the invention.

FIG. 3 illustrates histogram data produced by a flow cytometer for sperm stained at 25° C. with Hoechst 33342 for 3 hours

### DETAILED DESCRIPTION OF THE INVENTION

Referring now to FIG. 1, the invention relates to sorting populations of sperm and producing populations of viable sperm enriched in sperm carrying male or female chromosomal determinants of sex relative to the starting population wherein certain or even all of the steps between the step of sperm collection 10 and the step of preparing GES for transport and use 60 can be conducted using sperm maintenance media based on the same or substantially the same buffer system.

The function of a sperm maintenance medium considered broadly is to provide a suspension fluid meeting all of the energy, electrolyte, buffering, membrane stabilization, and other identified criteria for preserving and enhancing the viability and efficacy of the sperm used in producing GES. Thus, the sperm maintenance media can include all of the ingredients known in the art including energy source, electrolytes, buffer systems, plasma membrane stabilizers including proteins, lipids, lipoproteins, and other compounds (in an amount not intolerably interfering with sorting), and other ingredients, excluding only elements at each step that unacceptably interfere with that step of the process.

Previously it has been the practice to select the various media used in such processes based on the requirements of the individual steps or on general considerations relating to sperm maintenance. However, according to an aspect of the invention herein, the media are selected by determining an effective staining and maintenance medium for staining sperm using the QVDS stain, and then ensuring that the sperm maintenance media used at other steps in the process, including the sheath fluid for FACS, utilize the same or substantially the same buffer system and are consistent with effectively maintaining sperm viability. Thus, for example, in accordance with the invention, the same or substantially the same buffer system will be employed during the staining step as is used in the sheath fluid, and optionally the same buffer system will be used in one or more of the of the other steps such as in the initial diluent or extender used to dilute semen when collected or prior to sorting and in the steps following sorting. Illustrative buffer systems are shown in Table 1.

TABLE 1
CONCENTRATION OF BUFFER CONSTITUENTS (GRAMS/LITER)

| | | | | HEPES- | Na | | | | | |
|---|---|---|---|---|---|---|---|---|---|---|
| COMPONENTS | TRIS | TEST | BGM-1 | SALINE | CITRATE | CUE | CAPROGEN | IVT | TALP | |
| NaCl | — | — | 5.84 | 7.6 | — | — | — | — | 5.84 | (100 mM) |
| KCl | — | — | 0.23 | 0.3 | — | 0.4 | — | 0.4 | 0.23 | (3.1 mM) |
| NaHCO3 | — | — | 2.1 | — | — | 2.1 | — | 2.1 | 2.1 | (25 mM) |
| NaH2PO4 H2O | — | — | 0.04 | — | — | — | — | — | 0.04 | (.29 mM) |
| Na Lactate | — | — | 3.68 ml | — | — | — | — | — | 3.68 ml | (21.6 mM) |
| CaCl2 | — | — | 0.31 | 0.15 | — | — | — | — | 0.31 | (2.1 mM) |
| MgCl2 | — | — | 0.08 | 0.1 | — | — | — | — | 0.08 | (.4 mM) |
| Hepes | — | — | 2.38 | 2.38 | — | — | — | — | 2.38 | (10 mM) |
| Tris Base | 30.3 | 10.28 | — | — | — | — | — | — | — | |
| Citric Acid Monohydrate | 15.75 | — | — | — | — | 0.87 | — | — | — | |
| Na Citrate Dihydrate | — | — | — | — | 29 | — | 20 | 20 | — | |
| TES | — | 43.25 | — | — | — | — | — | — | — | |
| Fructose | 12.5 | — | — | 2.52 | — | — | — | — | — | |
| D-Glucose | — | 2 | — | — | — | 3 | 3 | 3 | — | |
| Steptamycin | — | 0.25 | — | — | — | — | — | — | — | |
| Penicillin-G | — | 0.15 | — | — | — | — | — | — | — | |
| Glycine | — | — | — | — | — | — | 10 | — | — | |
| Glycerol | — | — | — | — | — | — | 12.5 ml | — | — | |
| Caproic Acid | — | — | — | — | — | — | 0.325 | — | — | |
| Sulfacetamide | — | — | — | — | — | — | 0.1 | — | — | |
| Sulfanilamide | — | — | — | — | — | — | — | 3 | — | |
| BSA Frac V | | | | | | | | | 6 | |
| Pyruvate | | | | | | | | | .11 | (1 mM) |
| Gentamicin | | | | | | | | | .5 | ml |

Among the buffer systems that can be used in accordance with the invention are systems known to those skilled in the art for use with semen maintenance media, including but not limited to TES, TEST, Tris, BGM1, BGM3, HEPES-Saline, NaCitrate, CUE. Caprogen, IVT, and the like. While certain preferred buffer systems are described above in Table 1, the invention is not limited to those mentioned, but includes any known or hereinafter known to those skilled in the art in accordance with applicable legal principles that are used in accordance with the claimed invention.

According to a preferred aspect of the invention, the buffer system used is selected for the particularities of the GES being produced and the processes being used to produce the GES. Thus, certain buffer systems and pH values enhance solubilities of certain QDVS and may be preferred when those dyes are used. Therefore, according to the invention, the same or substantially the same buffer system will be used for other fluids used in GES production, including the sheath fluid, optionally excluding components that unacceptably interfere with the function of the sheath fluid. Thus, even though protein supplied by egg yolk, milk, serum albumin and the like may be desirable as a membrane stabilizer to prevent loss of protein, lipids, and other membrane components from sperm membranes or other purposes, it may be preferred to maintain the protein concentration in the sheath fluid during the flow sorting phase at a low level to prevent excessive interference with light stimulation and emission used during the sorting process. In such an instance, the sheath fluid composition can be selected to include the same buffer system and optionally to include other components such as ionic electrolytes, energy sources, membrane stabilizers and the like that do not intolerably interfere with the function of the sheath fluid.

Since in most instances, the sheath fluid is only briefly in contact with the sample fluid containing sperm during sorting, while after sorting the sheath fluid tends greatly to dilute the sample fluid in the collection chamber, either the sheath fluid can be adapted to include as many of the other components as possible for preventing the dilution effect, or the collection fluid can be supplemented with components excluded from the sheath fluid so as to provide an advantageous environment for the sperm after sorting.

A particularly preferred buffer system for sperm maintenance media is the TEST (TES-tris) buffered medium described in Table 1 above.

According to another preferred aspect of the invention, all of the steps from 20 to 50 preferably occur in a relatively narrow temperature range from above the thermotropic phase transition temperature of the sperm being sorted (for example, from about 17° C. for porcine sperm or more broadly from above 4° C. for bovine sperm) up to less than about 30° C. As used herein, the thermotropic phase transition temperature of the sperm is the temperature at or below which sperm of a given species experience cold shock due to membrane leakage. The thermotropic phase transition temperature is strongly influenced by species, being lower for bovine sperm than for porcine, and is also influenced by the sperm maintenance medium itself.

Referring again to FIG. 1, in a particular aspect, step 10 involves collecting semen from species such as mammals (not excluding humans) such as cattle, pigs, horses, sheep, deer, as well as others, where there is a significant difference in total chromosomal DNA (typically in the range of about 2.5% about 5%) depending on whether the X or Y chromosome is present. Thus, for example, it is possible to distinguish mammalian X-bearing and Y-bearing sperm based on difference in total chromosomal DNA present.

Semen of different species can be collected using methods known in the art. Collection methodologies and materials such as buffers, extenders, sheath fluids, and the like are well known and even commercially available. For mammals, for example, semen can be collected artificially using a gloved-hand method for the boar or artificial vagina for the males of other species mentioned above. Semen can also be collected from the males using electro-ejaculation methods.

After the semen has been collected into a collection vial, it can be prepared by step 20 for transportation optionally with QDVS staining of sperm. This step will frequently involve diluting the semen with an appropriate buffer or extender (preferably a selected sperm maintenance medium containing the same or substantially the same buffer system as will be used during staining that has been selected in accordance with the invention) that is used to extend the storage life or lifespan of the sperm outside the body as well as to confer additional benefits by virtue of selection for those advantages. These buffers themselves are often well known and reported in the scientific literature and the chemical composition of these buffers is adapted to the species of interest.

In accordance with a preferred aspect of the invention, the semen extender comprises a sperm maintenance medium comprising a buffer system that is common to or used in each step of production of GES. The function of the sperm maintenance medium is that of furnishing energy and nutrients to the stored sperm, provide buffering action to compensate for shifts in pH due to lactic acid formation, provide protection against rapid cooling and temperature shock, maintain the optimum osmotic pressure and balance of electrolytes including proteins for the media, inhibit the growth of microorganisms, and increase the volume of the original semen so that its use can be extended to many animals. For example, one collection of semen from a bull that is properly diluted can be used to AI from 300 to 800 cows and heifers.

Many semen extenders are known to those skilled in the art including those described in the patent literature cited above and incorporated by reference. Examples of extenders for cattle include 2.9% sodium citrate—egg yolk buffer (Salisbury et al., J. Dairy Sci., 24:905 (1941)). A particularly advantageous buffer for bulls is, for example, the HEPES buffer which can be prepared as described in J. J. Parrish, “Capacitation of Bovine Sperm by Heparin,” 39 Biology of Reproduction, 1171-1180 (1988). Addition of 0.1% BSA (bovine serum albumin) can also be advantageous. For boar sperm, similar extenders exist as diluents for artificial insemination using fresh semen, e.g., BTS, MR-A, Modena, and Androhep. There are many other commercially available diluents known to those skilled in the art that can be purchased with instructions for use as well as described in the relevant literature. The diluents facilitate manipulating the sperm cells in a laboratory to examine sperm morphology, concentration, functionality, activity, viability, etc.

To illustrate, for boars, the following buffer/extenders might be used: Acromax available from Insemination Technics and Supplies International, Inc. RR3, Princeton, Ontario, N.J.; VMD-Mulberry III available from V.M.D. n.v., Berendonk 74 B-2370 Arendonk, Belgium; BTS—chemical composition: glucose 37 g/l; sodium citrate dihydrate 6 g/l; EDTA 1.25 g/l; Sodium bicarbonate 1.25 g/l; potassium chloride 0.75 g/l; distilled water 1000 mL. pH 7.2; Modena—chemical composition: glucose monohydrate 27.5 g/l; sodium citrate 6.9 g/l; sodium bicarbonate 1.0; EDTA 2.35 g/l; Tris buffer 5.65 g/l; citric acid 2.9 g/l; 1000 mL distilled water; Androhep—chemical composition: glucose 26 g/l; sodium citrate 8 g/l; sodium bicarbonate 1.2 g/l; EDTA 2.4 gall BSA 2.5 g/l; HEPES 9.5 g/l; pH 6.8.

After or simultaneously with dilution of the sperm cells for handling and transport, the cells can also be contacted with a suitable QDVS under conditions including temperature and time of incubation effective for uniform staining. Where a common buffer system has already been used for extending or diluting the semen, this can be as simple as formulating the medium to contain a low level of the QDVS or by adding the QDVS in an appropriate solution to the common medium. In many instances, it will be possible to use QDVS as described below that will readily permeate the cells and nuclei and bind to the chromosomes. In other instances; it may be desirable to treat the sperm to facilitate permeation without unacceptably reducing viability or motility. Any suitable method known to those skilled in the art may be used. These methods can include electroporation, cell-permeation-enhancing solutions, e.g., mild surfactants, and the like. In yet other instances, it may be desirable to centrifuge the sperm and resuspend the centrifuged sperm in another medium, albeit based on the same or substantially the same buffer system to remove certain components (excessive glucose, egg yolk, etc.) of the suspension that may interfere with sorting by FACS.

According to the invention, the QDVS can be any nuclear staining dye that is cell-permeant or can be caused to be cell-permeant in the presence of the staining medium without unduly negatively affecting viability or efficacy of the sperm. The QDVS should be non-toxic in any appreciable degree to the sperm since once stained, the dye may remain with the cells until fertilization occurs. A particularly preferred dye is the bisbenzimide commercially available as Hoechst H33342 fluorochrome since it has low toxicity and is readily cell-permeant. This dye is particularly advantageous because fluorescence is dramatically enhanced after binding to DNA.

In accordance with a particular aspect of the invention, the bisbenzimide (bisbenzimidazole) can be modified by addition of a fluorophore that results in a fluorescence response by the conjugate to excitation by visible light. Preferably these conjugate molecules resemble the bisbenzimide molecule in that binding to DNA enhances their fluorescence. and represent an improvement over the bisbenzimide molecule in that the conjugates fluoresce in response to visible light.

Particularly preferred fluorophores are visible-light-excitable dipyrrometheneboron difluoride derivatives. Dipyrrometheneboron difluoride dyes are membrane permeant fluorescent compounds available from Molecular Probes Inc. under the BODIPY® trademark as described in, for example, U.S. Pat. No. 5,338,854 and U.S. Pat. No. 4,774,339 herein incorporated by reference. Preparation of an exemplary bisbenzimide-dipyrrometheneboron difluoride conjugate is described in Example 1 below. Other fluorophores of the class described in the preceding paragraph, such as, for example, fluoroscein and its derivatives may also be used.

Those skilled in the art will appreciate that such fluorophore-modified QVDS may be prepared by modifying or functionalizing the conjugate DNA stains with otherwise suitable properties so that they have sufficient solubility in the desired pH and temperature ranges. For example, chemical modifications can be made to enhance appropriate solubility by (1) changing the pKa of functional groups on the DNA stain, (2) adding an ionic solubility-enhancing group, either cationic or anionic, attached through an appropriate linker, or (3) adding nonionic solubilizing groups such as ethylene glycol or polyethylene glycol moieties.

Thus, within the scope of the invention, the bisbenzimide and visible wavelength fluorophore can be connected in many different ways. Example 1 illustrates one way they can be connected; however, persons skilled in the art can readily select many other ways of fluorophores and methods of connection. Supplies and consultation services to assist in such selection are readily available to those skilled in the art from commercial entities in the business of making and selling the fluorophores such as, for example, Molecular Probes Inc., 4849 Pitchford Ave., Eugene, Oreg. 97402

Preferably, the chemical entity linking the bisbenzimide to the visible wavelength fluorophore will be selected to not result in significant negative effects upon viability, solubility, stability, uptake, cell storage, flow cytometry, formulation, cost-of-goods or fluorescence properties. Preferably the chemical functionality of the inking entity will be selected to enhance properties such as stability, solubility, viability, uptake, cell storage, flow cytometry, formulation, cost-of-goods or fluorescence properties.

The use of the above mentioned conjugates allow the use of visible light excitation fluorescence, which has the advantage of being less damaging to the cells and DNA relative to UV excitation Since the energy level of the photons emitted in the visible light region have less energy than photons in the UV region and since the flux of photons through the bisbenzimide-visible wavelength fluorophore system will probably differ from the commonly used Hoechst 33342, it may become necessary to adjust or modify the flow cytometry detector system to be more sensitive and yet minimize the contributions from noise. It is also often possible to increase the emission signal strength by increasing the power of the laser. Both of these approaches are techniques that can help minimize instrument limitations on quantitating the difference in fluorescence from X and Y chromosome bearing sperm cells.

### EXAMPLE 1

### Bisbenzimide-BODIPY Conjugate

A bisbenzimide-BODIPY conjugate was prepared using commercially available starting materials as follows:

### a. Preparation of 9-[5-[5-(4-methyl-piperazinyl)-2-benzimidazolyl]-2-benzimidazolyl]phenoxy)octan-1-oic acid, istrifluoroacetic acid salt—see structure 1 below

Under a nitrogen atmosphere, 660 μL of a hexanes solution of lithium-t-butoxide (1.0M) was added to a solution of 70.4 mg of p-[5-[5-(4methyl-1-piperazinyl)-2-benzimidazolyl)-2-benzimidazolyl]-trihydrochloride phenol (commercially available as Hoechst 33258) in 2.5 mL of anhydrous DMSO. 8-Bromooctan-1-oic acid (30.4 mg) was then added and the mixture stirred at room temperature for 18 hours. Reverse phase HPLC purification of the reaction mixture utilizing 0.1% trifluoroacetic acid in the mobile phase yielded 20.8 mng of 1 (17%).

Mass spectra: M+H⁺=567 m/z.

### b. Preparation of N-(3-aminopropyl)-8-(p-[5-[5-(4-methyl-1-piperazinyl)-2-benzimidazolyl-2-benzimidazolyl]phenoxy)octan-1-amide, tetrafluoroacetic acid salt—see structure 2 below

Under a nitrogen atmosphere, 65 μL of a DMF solution of O-benzotriazol-1-yl-N,N,N′N′-tetramethyluronium hexafluorophosphate (0.100M) and 320 μL of a DMF solution of diisopropylethyl amine (0.100M) were added to a 250 μL DMF solution containing 5.75 mg of 1. After 35 minutes, the solution above was added to a 500 μL DMF solution containing 10 μL of 1,3-diaminopropane. The mixture was stirred 20 minutes at room temperature. Reverse phase HPLC purification of the mixture yielded 5.73 mg of 2 (84%). Mass spectra: M+H⁺=623 m/z.

### c. Preparation of [2-[(3,5-dimethyl-1H-pyrrol-2-yl-kN)methylene]-N(N-(N-(8-(p-[5-[5-(4-methyl-1-piperazinyl)-2-benzimidazolyl]-2-benzimidazolyl]phenoxy)octan-1-oyl)-3-aminopropyl)hexan-6-amide-2H-pyrrole-5-propanamidato-kN1]difluoroboron, trifluoracetic acid salt—see structure 3 below

Under a nitrogen atmosphere, 200 μL of a 0.010M DMF solution of [2-[(3,5-dimethyl-1H-pyrrol-2-yl-kN)methylene]-N-(5-carboxypentyl)-2H-pyrrole-5-propanamidato-kN1]difluoroboron, N-hydroxysuccinamide ester (commercially available as BODIPY FL-X,SE from Molecular Probes Inc.) was added to a 500 μL DMF solution of 1.98 mg of 2 and 3.4 μL of diisopropylethyl amine. The reaction mixture was purified by reverse phase HPLC after stirring at room temperature for 2 hours to yield 2.26 mg of 3 (91%). Mass spectra M+H⁺=1010 m/z.

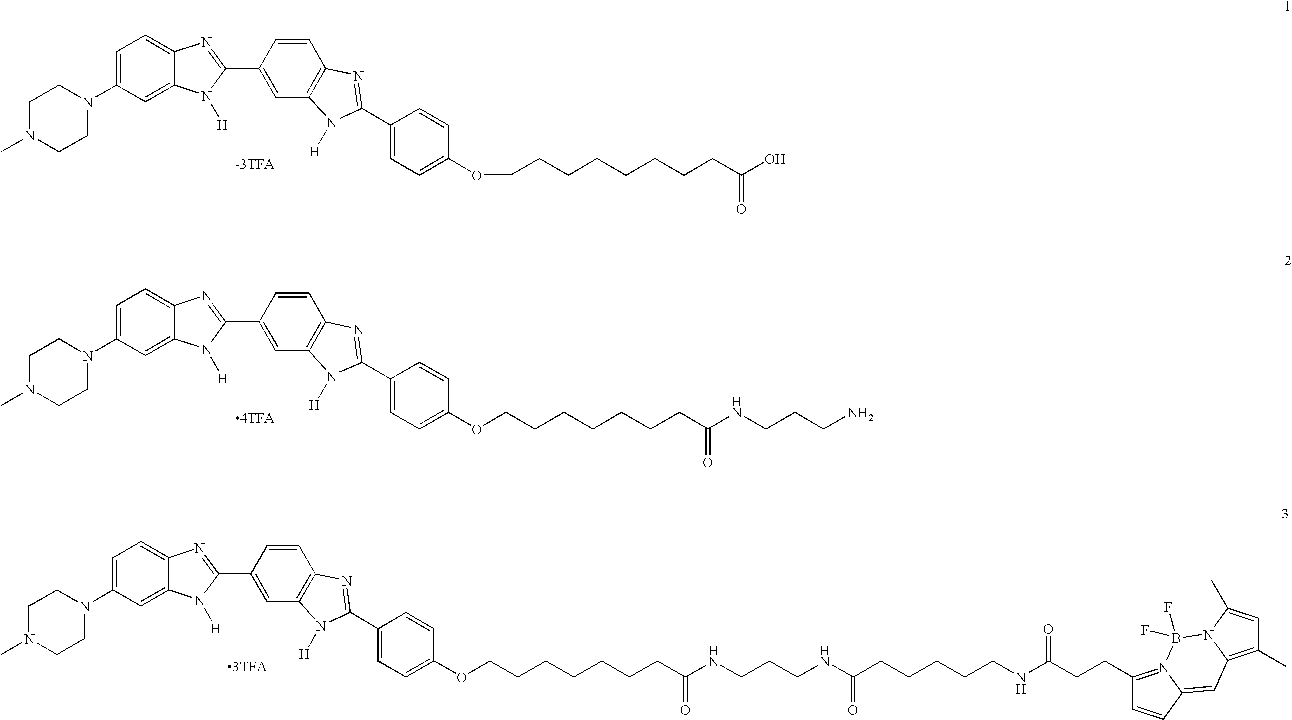

In accordance with the invention, sperm in semen, preferably extended with an extender or diluent are contacted with QDVS dyes under conditions including temperature and incubation time effective for completely and quantitatively staining DNA in the sperm.

According to an aspect of the invention, the temperature of incubation is in the range of about 15° C. to less than about 30° C. More preferably, the temperatures are in the range of about 18° C. to about 25° C. since these temperatures are preferred for handling and shipping of sperm and are near ambient temperature as used in sperm sorting facilities. An amount of QDVS dye consisting of H33342 or of the bisbenzimide—BODIPY conjugate can be added in the range of about 4 to about 5 μg/ml, more preferably about 5 μg/ml since such concentrations are known to be effective for staining (see Johnson et. al., 1999). It will be appreciated that the concentration may need to be varied depending on the concentration or density of sperm in the semen being contacted with the dye; however, such adjustment can be readily made by persons skilled in the art. The optimal amount of stain for most species has been reported to be about 40 micrograms per 150×10⁶ sperm. See, for example, L. A. Johnson and Glenn Welch, “Sex Preselection: High Speed Flow Cytometric of X and Y Sperm for Maximum Efficiency.” 52 Theriogenology 1323-1341 (1999).

The QDVS-sperm mixture can then be incubated for an effective period, for example, from a lower limit of about 1 hour since effective staining under appropriate conditions of temperature and pH can be achieved with that incubation period to about 18, 24 or more hours since overnight or express delivery of stained sperm to a sorting facility can be expected to occur in that period of time.

According to an aspect of the invention the flow cytometer can be adjusted to enable the excitation and detection of light emitted in the visible light range (e.g. emission above ˜480 nM wavelength). If one assumes the use of an Epics 751 (Coulter Corporation) having a typical 5-watt argon ion laser such as the Coherent model 306 laser, then the following steps should be taken to switch from multi-line ultra-violet (UV) excitation to visible excitation at a wavelength of 488 nM. Such steps are merely illustrative since persons skilled in the FACS arts will readily perform such steps customized for the particular fluorophore selected.
1. Change optics (high reflector, output coupler) in laser so that gain cavity is now appropriate for the 488 nm line.
2. Change magnet current to the low setting to reduce constriction of plasma in the plasma tube.
3. Change the aperture of the gain cavity to ensure TEM₀₀ mode resonance in the cavity.
4. Re-align and possibly change beam-shaping optics to accommodate the longer wavelength light. They are no typically achromatic optics.
5. Change optical filters in front of the PMTs (photo multiplier tubes) to select for the BODIPY emission and to block the 488 nm laser emission
6. Adjust the focus or the fluorescence collection optics to optimize for BODIPY emission.
7. Adjust detector sensitivity and amplifier gain to center the measured fluorescence of the cells on scale. Adjustment may be up or down depending on the characteristics of the detector. Adjustments include PMT high voltage and amplifier gain.
8. Adjust optical alignment using nuclei to optimize instrument alignment.

These steps, of course, only involve the switching of one to the other on a water-cooled, argon ion laser that supports both wavelengths. However, if the technician chose BODIPYFLX (FITC like) as the dye of choice to be using all the time, then a different laser would be chosen. For example, a Neodymium-based solid state laser might be chosen in place of the Argon ion laser as it offers low noise, low heat output, compact size, low cost, and a higher working efficiency (˜30% vs. 0.1% for Argon ion). Then perhaps few if any of the above steps would need to be conducted on a routine basis.

If one assumes the use of a MoFlo (Cytomation, Inc.) having an Argon laser, then the following steps should be taken to switch from multi-line ultra-violet (UV) excitation to visible excitation at a wavelength of 488 nM:
a. Install 515LP collection optics.
b. Change laser line; (install visible optics, output coupler and high reflector, adjust prism to select 488 nm).
c. Perform basic instrument laser alignment
d. Install visible light laser focus lens.
e. Calibrate using Coulter Flow Check beads to obtain 1% CV's.

In most cases, it is believed that it will not be necessary to treat the sperm to facilitate QDVS uptake and binding. However, if desirable, the sperm may advantageously be treated to facilitate entry of the QDVS or its conjugates into the cells. For example, chemical shock or cell-permeation-enhancing solutions may be used to facilitate uptake, for example, using DMSO (dimethylsulfoxide) or glycerol or the like. Cells having stain efflux systems might be treated with compounds that inhibit this system. For example, classes of calcium channel blockers such as verapamil, trifluoperazine and others (DNP, novobiocin). Also, compounds that might inhibit the polyamine biosynthesis pathway could enable uptake of stain. For example, difluoromethyl ornithine (DFMO) has been shown to enhance the uptake of polyamines in mammalian cells. Where it is desired or advantageous to use other or more stringent techniques, such treatments can include use or liposomes or many of the techniques that are used by those skilled in the art to introduce stains, dyes, genes or vectors into living cells. These methods include, but are not limited to: microinjection such as used by Gordon et al. 1980, Proc. Natl. Acad. Sci.: 7380-7384 and since extended to rabbits, sheep, cattle and pigs; electroporation; DEAE-dextran-mediated transfer; coprecipitation with calcium phosphate, and other techniques.

Following preparation and optionally staining of the sperm as described herein, the extended semen can be transported to a semen sorting facility as indicated by step 30 in FIG. 1. At the sorting facility, the semen can be prepared for flow cytometry, for example, at ambient temperatures as is known in the art. This may involve additional or different buffers or extenders, such as BTS (Beltsville Thaw Solution), Androhep, MODENA, Acromax, Vital-boar, X-Cell, Mulberry III, and the like, as well as those shown in Table 1. It may be advantageous to avoid egg yolk or milk buffers prior to sorting. If the sperm were not stained prior to shipment, the staining as described hereinabove can occur in the sorting facility. After preparation, sorting by flow cytometry occurs as illustrated by reference numeral 50 in FIG. 1.

Persons skilled in the art will appreciate that by practice in accordance with the invention, all of steps 20-50 of FIG. 1 can occur above the thermotropic phase transition temperature Tm for sperm of the species being sorted, for example, in the temperature range of above about 4° C. (for bovine sperm) or above about 17° C. for porcine sperm to less than about 30° C. and more preferably in the range of about 18° C. to about 25° C. During the flow cytometry step as shown in U.S. Pat. No. 5,135,759 and U.S. Pat. No. 5,985,216, incorporated herein by reference for flow cytometric methods and apparatus, the sperm are preferably subjected to hydrodynamic forces which cause the sperm (typically flattened in structure) to be more uniformly oriented for fluorescence stimulation by the light source. It is expected that use of lower temperatures as described herein during the step(s) immediately preceding the sorting step, will result in sperm having a low rate of motility which can allow more uniform orientation by the hydrodynamic forces resulting in an advantageous efficiency and purity of separation ass compared to sorting of sperm after the relatively high temperature separation step of U.S. Pat. No. 5,135,759.

Preferably the flow cytometry techniques are such as not adversely to affect either motility or viability of the cells, as they are being analyzed and sorted. As indicated, suitable such techniques are described, for example, in U.S. Pat. No. 5,135,759 and U.S. Pat. No. 5,985,216 that are incorporated herein by reference for this purpose.

Preferably, the stained sperm sample subjected to flow cytometry will have a fluorescence absorber to absorb fluorescence due to dead sperm. A suitable quencher can be made using FD&C#40 stock at 25 mg/ml (in dH2O), of which 1.0 μl can be added to 1 mL of sperm solution and held at ambient (23° C.-25° C.) for 5 min to allow dampening of the fluorescence due to dead sperm.

The sheath fluid buffer used during cytometry can be any suitable buffer that is nontoxic to the sperm and does not interfere with flow cytometry. For general use, a preferred sheath fluid is PBS (phosphate buffered saline) with 0.1% BSA and 0.1% EDTA (wt/volume) at a pH of 7.2. Antibiotics are added to the sheath fluid (100 μg/ml penicillin G and 75 μg/ml streptomycin) and the sheath fluid is sterile-filtered. See, e.g., Rath D. et al., “In vitro production of sexed embryos for gender preselection: high speed sorting of X-chromosome bearing sperm to produce pigs after embryo transfer”, J. Animal Science 77:3346-3352 (1999). For applications in accordance with certain aspects of the invention, the sheath fluid may contain the same or substantially the same buffer system as is used during the staining step optionally with some additional components being present. In any event, the sheath fluid will be substantially isotonic with the sample fluid.

### EXAMPLE 2

### Low Temperature Staining of Bull Sperm with Hoechst H33342 Followed by X,Y-Sorting

Bull sperm in citrate buffer at pH 6.9-7.0 is sent from collection facility to sorter facility by same-day delivery at 18° C. Upon receipt the sperm is divided into three portions and stored and stained overnight with Hoechst 33342 dye at 18° C., 20° C., or 22° C. (all in citrate buffer at pH 6.9). Each is checked at O hours (after overnight staining) for separation into X- and Y-sperm by flow cytometry, then the temperature is allowed to rise to 24° C. to enhance uptake. At 1.5 and 5 hours, the samples are checked again for separation into X- and Y-bearing sperm. The results are shown in the following Table 2.

TABLE 2
Results of staining bull sperm overnight with Hoechst 33342 (HO) at
18° C., 20° C. or 22° C. evaluated for separation of Y- and X-bearing
sperm by flow cytometry after warming to room temperature
for various periods of time

| | 0 hr | 1.5 hr | 5.0 hr |
|---|---|---|---|
| | incubation @ | incubation @ | incubation @ |
| Treatment | RT | RT | RT |
| 18° C. o/n with HO | no separation | no separation | no separation |
| 20° C. o/n with HO | no separation | no separation | no separation |
| 22° C. o/n with HO | no separation | close to | separation into X |
| | | separation | and Y populations |

These data indicate that temperature of sperm storage during staining influences the efficiency of uptake of HO dye at room temperature (24° C.). Since the coefficient of variation indicated that the sperm were close to separation at 1.5 hours incubation at room temperature, it is believed that a 3-hour incubation at room temperature would suffice for separation. These results indicate that effective separation of X- and Y-bearing sperm can be achieved within reasonable periods of time at room temperature. Since at 0 hours after overnight incubation with HO dye, the motility of the various treatments did not show any apparent significant difference, the results of this run also indicated that prolonged exposure to the HO dye medium does not compromise sperm viability.

### EXAMPLE 3

### Low Temperature Staining of Bull Sperm with Hoechst H33342 Followed by X,Y-Sorting

Bull semen was collected from a sexually mature bull using an artificial vagina and the sample was diluted with citrate buffer (pH 7.0) at 1 part semen: 3 parts buffer. The sample was transferred to the flow cytometry laboratory at 18° C. The concentration of the sample is determined using a hemocytometer and the cells were diluted with an appropriate amount of TEST buffer (pH 7.35) to obtain 100 million sperm per mL. Ten microliters of a stock concentration (5 mg/ml in dH₂O) of Hoechst 33342 was added to the sample of sperm and the cells were incubated at 25° C. for up to 4 hours. A second population of cells was handled in like manner but was incubated at 35° C. for 1 hour to serve as a positive control. A third population of cells was handled in like manner but the buffer pH is 7.2 instead of pH 7.35 to determine if buffer pH influences uptake of Hoechst 33342. At one-hour intervals for up to 4 hours, 200 μL micro aliquots were removed from the samples and evaluated using a Coulter Epics flow cytometer. The split index information was collected for each sample per treatment group. The data is shown in the Following Table 3.

TABLE 3

| | 1 h Split | 2 h split | 3 h split | 4 h split |
|---|---|---|---|---|
| Treatment | index* | index | index | index |
| 35° C. control | 30% | NA | NA | NA |
| 25° C. in TEST pH 7.35 | 0% | 0% | 10% | 30% |
| 25° C. in TEST pH 7.2 | 0% | 0% | 0% | 0% |

*Split index is a semi-quantitative index for determining the resolution of the X and Y chromosome-bearing sperm populations and is calculated by measuring the depth of valley between the two peaks representative of the X-bearing and Y-bearing sperm populations. In general, a 5% or greater split index is a good indication saturation of DNA with the dye has occurred in a significant subpopulation of sperm and that separation of X-bearing and Y-bearing sperm can be achieved.

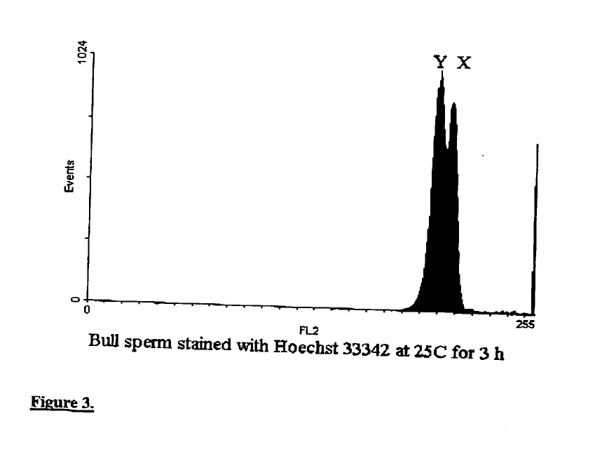

FIG. 3 depicts histogram data produced by the flow cytometer for sperm stained at 25 C with Hoechst 33342 for 3 hours.

These results indicate that pH influences the efficiency of Hoechst (HO) uptake into living sperm cells and that a pH of 7.3 or higher can be used to advantage with the Hoechst dye as compared with lower pH values. The results also support the earlier (see previous Example) that a 3-hour period of room temperature incubation (after overnight incubation at a lower temperature in the presence of the dye) can be effective for achieving separation.

### EXAMPLE 4

### Low temperature Hoechst Staining of Bull Sperm Followed by X, Y-Sorting

Bull sperm are collected at a collection facility, extended in citrate buffer pH 6.9 and sent at 18° C. by overnight express mail to a sorting facility. The sample is divided into two portions, centrifuged to separate sperm from supernatant, and each portion resuspended respectively in citrate buffer and HEPES buffer, both at pH 7.4. 0.1% BSA is present in the HEPES buffer. Hoechst dye is added to each sample, both samples are allowed to warm to 24° C. to enhance stain uptake and are checked by flow cytometry at 2.5 hours for separation into X- and Y-bearing sperm. The results are shown in the following Table 4.

TABLE 4
Results of staining bull sperm with Hoechst 33342 (HO) at 24° C.
evaluated by cell cytometry after 2.5 hours

| | Sort results after 2.5 h incubation |
|---|---|
| Treatment | at room temperature (24° C.) |
| Sperm in NaCitrate buffer | No separation into X and Y |
| Sperm in HEPES buffer | Sperm were separated into X and Y |

These data indicate that buffers play a role in the uptake of HO dye at room temperature. The data also indicate, by comparison to Example 2, that incubation overnight in the presence of the dye may be more important for some buffer systems, such as citrate, than for others. For this reason, it may be desirable to exclude citrate buffer from some media prepared in accordance with the invention.

### EXAMPLE 5

### Staining of Bull Sperm Nuclei and Intact Sperm with a Bisbenzimide-BODIPY Conjugate Followed X,Y-Sorting

Bull semen was collected from a sexually mature bull using an artificial vagina and the sample was diluted with citrate buffer (pH 7.0) at 1 part semen: 3 parts buffer. The sample was transferred to the flow cytometry laboratory at 18° C. (within 2 h from semen collection). The concentration of the sample was determined using a hemocytometer and the living cells were diluted with an appropriate amount of TEST buffer (pH 7.35) to obtain 100 million sperm per mL. Ten microliters of a stock concentration (5 mg/ml in dH₂O) of a bisbenzimide-BODIPY conjugate was added to the sample of sperm and the cells were incubated at 35° C. for 1 hour and then stored at room temperature for up to 3 hours (total of 4 hours exposure to dye). For preparation of sperm nuclei, an aliquot of the original semen sample (above) was sonicated and the nuclei placed into a 1.5 mL microcentrifuge tube and brought up to a final volume of 1 mL using PBS buffer. The final concentration of sperm nuclei per tube was 10 to 15 million sperm. The nuclei were then stained using 2 μl of the bisbenzimide-BODIPY conjugate stain. The tube was then incubated at 35° C. for 1 h prior to evaluation on the flow-sorting instrument. Prior to evaluation of sperm nuclei and intact sperm, samples were transferred into a plastic tubes as used for the flow sorter. At one-hour intervals for up to 4 hours, the samples were evaluated using a high-speed MoFlow flow sorter at a USDA facility. The split index information was collected for each sample. The data is shown below in Table 5.

TABLE 5
Sorting bull sperm into X and Y nuclei following staining with a
bisbenzimide-BODIPY conjugate and using
488 nM visible light excitation.

| | Laser | | | | | |
|---|---|---|---|---|---|---|
| | Wave- | Laser | | 90° | | |
| Sample | length | power | 90° | PMT | | Split |
| ID | (nM) | (mW) | Filter | (Volt) | CV | index |
| Bull | 488 | 500 | 530 LP | 356 | 2.37 | 35% |
| nuclei | | | | | | |

These results indicate that visible light (488 nm) can be used to excite a Hoechst-derivative dye and can be used for separation into X-bearing and Y-bearing sperm nuclei.

Living bull sperm were separated under the same conditions except that sperm were incubated in both TEST and TALP buffers, and FD&C 40 was used to stain dead sperm to facilitate exclusion from collected GES. The results are shown in the following Table 6.

TABLE 6
Sorting bull sperm into X and Y-bearing populations following staining
with a bisbenzimide-BODIPY conjugate and using
488 nM visible light excitation.

| | Laser | | | | | |
|---|---|---|---|---|---|---|
| | Wave- | Laser | | 90° | | |
| Sample | length | power | 90° | PMT | | Split |
| ID | (nM) | (mW) | Filter | (Volt) | CV | index |
| Intact | 488 | 400 | 515 LP | 388 | 2.52 | 20% |
| bull | | | | | | |
| sperm | | | | | | |
| in | | | | | | |
| TEST | | | | | | |
| Intact | 488 | 500 | 530 LP | 356 | 2.66 | 3-5% |
| bull | | | | | | |
| sperm | | | | | | |
| in | | | | | | |
| TALP | | | | | | |

The results show that a visible-light-excited fluorophore (Hoechst-BODIPY conjugate) can be used to stain DNA of live sperm and be excited using visible light excitation (488 nM) to facilitate separation into X and Y bearing sperm subpopulations. The results also show that the TEST buffer system enabled more efficient uptake of the Hoechst-BODIPY conjugate facilitating improved sorting efficiency/yield.

### EXAMPLE 6

### Hoechst 33342 and Bisbenzimide-BODIPY Conjugate Buffer Solubilities

Hoechst 33342 and the Bisbenzimide-BODIPY conjugate prepared in Example 1 were separately dissolved in DMSO to make 10 millimolar stock solutions. One microliter (μL) of the stock solution was then added to 110 μL of aqueous buffer solution placed in an HPLC (high performance liquid chromatography) vial and mixed by inverting a number of times. The samples were allowed to stand for 30 minutes and then centrifuged for 30 minutes. The centrifugation is required to deposit suspended material that could interfere with the analysis to the side of the vial. The solutions were then analyzed by HPLC by sampling the solution without touching the vial bottom or sides. Shown below are the measured quantities remaining in solution in the various buffers. Based on the procedure used, the maximum solubility that was measured for was 90 micromolar.

| | | |
|---|---|---|
| pH | Hoechst 33342 | Bisbenzimide-BODIPY conjugate |

TES Buffer

| | | | |
|---|---|---|---|
| 6.85 | 79 μM | 76 | μM |
| 6.98 | 80 μM | 38 | μM |
| 7.19 | 78 μM | ˜1 | μM |
| 7.34 | 75 μM | <1 | μM |
| 7.54 | 57 μM | <1 | μM |

Tris Buffer

| | | | |
|---|---|---|---|
| 6.76 | 34 μM | <1 | μM |
| 6.95 | 42 μM | <1 | μM |
| 7.15 | 49 μM | <1 | μM |
| 7.36 | 22 μM | <1 | μM |
| 7.53 | 15 μM | <1 | μM |

HEPES Buffer

| | | | |
|---|---|---|---|
| 6.85 | 79 μM | ˜1 | μM |
| 7.02 | 73 μM | ˜2 | μM |
| 7.23 | 27 μM | <1 | μM |
| 7.36 | 23 μM | ˜1 | μM |
| 7.52 | 36 μM | ˜1 | μM |

These results indicate that both Hoechst 33342 and the bisbenzimide-BODIPY conjugate have better solubility properties in the TEST buffer.

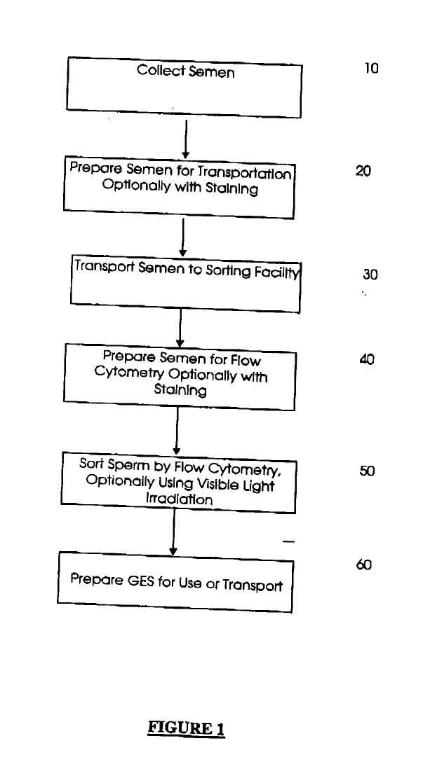

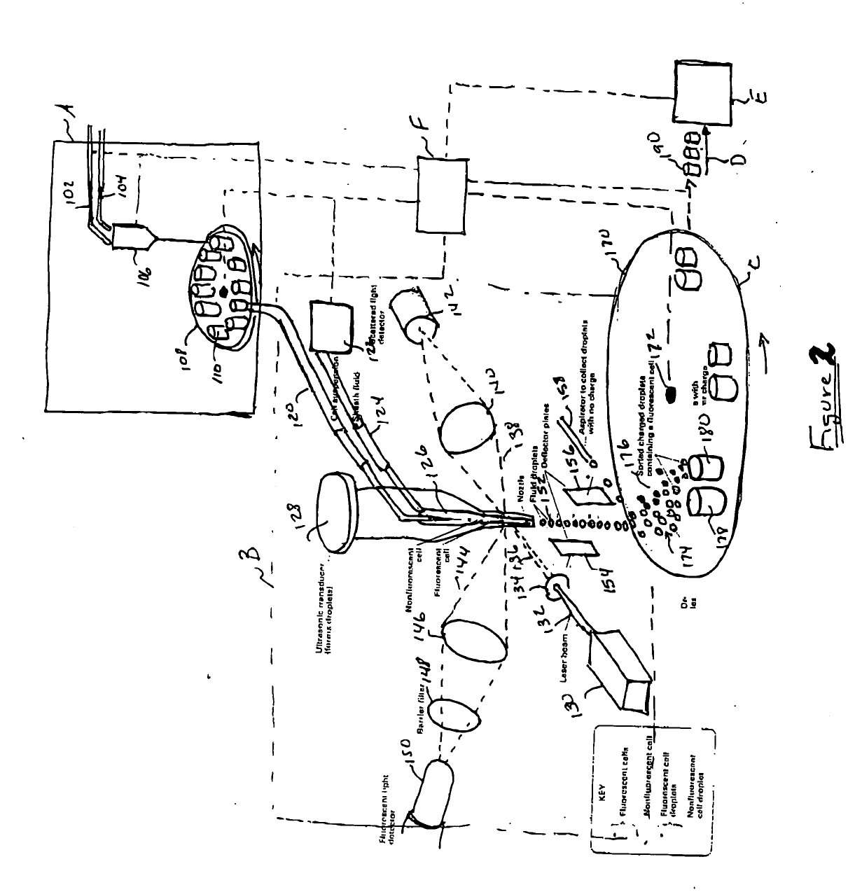

Referring now to FIG. 2 in detail, FIG. 2 illustrates schematically a flow cytometry system such as may be used for effecting separations based on fluorescence in accordance with the invention. FACS systems such as those described and referred to in U.S. Pat. No. 5,135,759, U.S. Pat. No. 5,985,216, U.S. Pat. No. 6,263,745 11, WO 01/37655, U.S. Pat. No. 6,149,867, U.S. Pat. No. 6,071,689, and WO 99133956, incorporated herein by reference, can be used. Preferably, such systems are modified as illustrated in FIG. 2 to automate the operation. Thus, the illustrated flow cytometry system includes preparation zone A, cytometry zone B, collection zone C. transfer zone D, and storage zone E all under automated control by controller F.

In preparation zone A as illustrated, a supply of sperm indicated at 102 and a diluent indicated at 104 are provided to constant temperature mixing zone 106 to provide diluted sperm which can be dispensed into containers 110 on rotating table 108 for sequential positioning and delivery, for example, via line 120 to cytometry zone B.

Cytometry zone B illustrates a conventional flow cytometry system in which sample fluid provided by line 120 and sheath fluid via line 124 are introduced into nozzle 126 controlled by droplet transducer 128, for example, an ultrasonic droplet transducer, to produce droplets 152 containing predominantly only one cell or cell cluster per droplet. Laser 130 provides laser excitation 132, which may be ultraviolet or preferably visible, and 136 via filter 134 to induce differential fluorescence in cells or cell clusters depending On the presence or absence of fluorophores therein. Filters 146 and 148 focus fluorescence 144 on detector 150. Scattered light 138 is focused by filter 140 on detector 142.

As droplets 152 leave nozzle 126, deflector plates 154 and 156 cause the droplets that have a negative charge proportional to fluorescence to be preferentially sorted into streams 174 and 176 and thence into collection vials 178 and 180 on turntable 170 of collection zone C. Vials 190 enriched or depleted in a target DNA sequence of interest are then moved via transfer zone to storage zone E where the individual samples are cryopreserved and maintained.

Referring again to FIG. 2, reference numeral 126 illustrates the use of a flow cytometer nozzle in the methods of the invention. As those skilled in the flow cytometry arts will appreciate, the nozzle must be sized appropriately for the class of cells or cellular cluster of interest. Such sizing is a matter of ordinary skill and need not be further described here. For separation of sperm that characteristically have flattened heads, it has been found advantageous to use nozzles that orient the sperm prior to detection. For example, a tapered needle can be used or a specially designed nozzle such as that illustrated in Rens et al., U.S. Pat. No. 5,985,216 which is incorporated herein by reference, with particular reference to FIGS. 1, 2 and 3 and corresponding text.

Although the invention has been described herein in terms of particularities of processes and compositions of matter and apparatus, the invention is not limited thereto but to the scope of the claims appended hereto, interpreted in accordance with applicable principles of law. Those skilled in the art will be enabled by the use of ordinary skill in the art in view of the teaching herein to provide many other processes and compositions of matter and apparatus useful for practicing the invention in its various aspects.

1. A method for sorting semen containing predominantly living, viable sperm into GES (gender enriched sperm) comprising:
a) staining DNA in the sperm for an incubation period effective for staining nuclei of living sperm cells sufficient to distinguish chromosomal determinants of sex in individual sperm based on resulting fluorescence under a fluorescence stimulating light source using a quantitative DNA vital stain (QDVS) under conditions selected from the following and combinations thereof: (a) temperature in the range from about 18° C. to less than about 30° C., (b) pH in the range of about 7.1 to about 7.6 (c) incubation for an extended period at a lower temperature followed by a shorter period at higher temperature to enhance staining; and
b) separating the thus-incubated sperm into GES using FACS (fluorescence-activated cell sorting and producing GES consisting predominantly of living sperm of which greater than 90% are of one sex.

2. The method of claim 1 wherein the temperature of staining is in the range of about 18° C. to about 25° C. and the pH of staining is in the range of about 7.3 to about 7.5.

3. The method of claim 1 wherein the QDVS is capable of fluorescence under visible light.

4. The method of claim 1 wherein the QDVS is selected from the group consisting of bisbenzimide and bisbenzimide labeled with a fluorophore capable of fluorescence under stimulation by visible light.

5. The method of claim 1 wherein the incubation period is in the range of about 1 to about 24 hours.

6. The method of claim 1 wherein the incubation period one hour or less.

7. The method of claim 1 wherein semen is contacted with QDVS shortly after semen collection and at least part of the incubation period occurs during transit from a semen collection facility to a semen sorting facility.

8. The method of claim 1 wherein incubation with QDVS at a temperature in the range of about 18° C. to less than about 30° C. is followed by flow cytometry at ambient temperatures.

9. The method of claim 2 wherein the semen is maintained at a pH less than about 7.1 prior to staining with QDVS at a pH in the range of about 7.1 to about 7.6.

10. The method of claim 2 wherein the semen are maintained during staining at a pH in the range of about 7.3 to about 7.5.

11. A process for producing GES (gender enriched semen) comprising:
a) providing a suspension of viable sperm produced from collected semen ejaculate that is extended and transported to a sorting facility;
b) staining the sperm using a QDVS (quantitative DNA vital stain) in the presence of a medium comprising a buffer system and further optionally including other components, the medium effective for maintaining viability of at least a portion of the semen;
c) producing at least one of X-enriched and Y-enriched GES based on the extent of QDVS staining;
d) collecting the resulting GES;
e) wherein steps c) and d) occur in the presence of media comprising at least said buffer system, and
f) partitioning the collected GES into dosage quantities for use or shipment.

12. The method of claim 11 wherein all of steps a) through completion of e) occur in the presence of media comprising at least said buffer system.

13. The method of claim 11 wherein step c) is conducted using a FACS (fluorescence-activated cell sorter) and the FACS utilizes a sheath fluid comprising said buffer system.

14. The method of claim 11 wherein said buffer system is selected based on efficacy of performance for staining and maintaining viability of sperm during staining of the sperm using the QDVS.

15. The method of claim 11 wherein said buffer system comprises a TEST (N[Tris(hydroxymethyl)methyl]-2-aminoethanesulfonic acid—tris-hydroxymethylaminomethane) buffer system.

16. The method of claim 11 wherein 11 wherein step c) is conducted using a FACS (fluorescence-activated cell sorter) and the FACS utilizes a sheath fluid comprising said buffer system and said buffer system comprises a TEST (N[Tris(hydroxymethyl)methyl]-2-aminoethanesulfonic acid—tris-hydroxymethylaminomethane) buffer system.

17. The method of claim 11 wherein all of steps a) through f) are conducted at a temperature between the between about the thermotropic phase transition temperature Tₘ of the membranes of the sperm being sorted up to less than about 39° C. and at an effective pH between about 6.8 and about 7.6.

18. The method of claim 11 wherein all of steps a) through f) are conducted at a temperature between the between about the thermotropic phase transition temperature Tₘ of the membranes of the sperm being sorted up to less than about 30° C. and at an effective pH between about 6.8 and about 7.6.

19. The method of claim 11 wherein all of steps a) through f) use media comprising said buffer system.

20. The method of claim 19 wherein all steps are conducted at a temperature between about the thermotropic phase transition temperature Tₘ of the membranes of the sperm being sorted up to less than about 39° C. and at an effective pH between about 7.3 and about 7.5.

21. A process for producing GES (gender enriched semen) comprising:
a) providing a suspension of viable sperm produced from collected semen ejaculate that is extended and transported to a sorting facility;
b) staining the sperm using a QDVS (quantitative DNA vital stain);
c) based on the extent of QDVS staining producing at least one of X-enriched and Y-enriched GES;
d) collecting the resulting GES; and
e) partitioning the collected GES into dosage quantities for use or shipment;
f) wherein from step a) starting after collection of the semen ejaculate until completion of step e) all steps occur at a temperature in a range from above the thermotropic phase transition temperature Tₘ of the membranes of the sperm being sorted up to less than about 30° C. and in the presence of a buffer system which is used at least for steps b), c) and d) and at an effective pH between about 6.8 and about 7.6.

22. The method of claim 21 wherein step c) is conducted using a FACS (fluorescence-activated cell sorter) and the FACS is operated at ambient temperature.

22. A process for producing GES (gender enriched semen) comprising:
a) providing a suspension of viable sperm produced from collected semen ejaculate that is extended and transported to a sorting facility;
b) staining the sperm using a QDVS (quantitative DNA vital stain) that fluoresces in response to visible light irradiation;
c) based on the extent of QDVS staining producing at least one of X-enriched and Y-enriched GES:
d) collecting the resulting GES; and
e) partitioning the collected GES into dosage quantities for use or shipment;
f) wherein from step a) starting after collection of the semen ejaculate until completion of step e) all steps occur (i) at a temperature in a range from above the lower semen viability temperature to less than the upper semen viability temperature and (ii) in the presence of sperm maintenance media effective for maintaining viability of at least a portion of the semen throughout the process.

23. The method of claim 21 wherein step c) is conducted using a FACS (fluorescence-activated cell sorter) and the FACS utilizes visible light irradiation for exciting fluorescence from stained sperm.

24. The method of claim 21 wherein the QDVS comprises a bisbenzimide modified by addition of a fluorophore that results in a fluorescence response by a resulting conjugate to excitation by visible light.

25. The method of claim 21 wherein the QDVS comprises a bisbenzimide-dipyrrometheneboron difluoride conjugate.

26. The method of claim 22 wherein stained sperm are irradiated with light at 488 nm.

27. The method of claim 21 wherein all steps occur at a temperature in a range from above the thermotropic phase transition temperature Tₘ of the membranes of the sperm being sorted up to less than about 30° C.

28. The method of claim 21 wherein all steps occur in the presence of a buffer system which is used at least for steps b), c) and d) and at an effective pH between bout 6.8 and about 7.6.

29. The method of claim 28 wherein said buffer system comprises a TEST N[Tris(hydroxymethyl)methyl]-2-aminoethanesulfonic acid—tris-hydroxymethylaminomethane) buffer system
